(19) United States
(12) Patent Application Publication
(10) Pub. No.: US 2026/0027379 A1
(43) Pub. Date: Jan. 29, 2026

(54) THERAPEUTIC PULSE GENERATORS

(71) Applicant: Medtronic, Inc., Minneapolis, MN (US)

(72) Inventors: Mark E. Viste, Brooklyn Center, MN (US); Paul Brian Young, New Richmond, WI (US)

(21) Appl. No.: 19/150,010

(22) PCT Filed: Jan. 8, 2024
(86) PCT No.: PCT/IB2024/050159

§ 371 Date: Jul. 22, 2025

Related U.S. Application Data
(60) Provisional application No. 63/441,076, filed on Jan. 25, 2023.

(51) Int. Cl. A61N 1/39

(52) U.S. Cl. CPC A61N 1/3981; A61N 1/3956

(57) ABSTRACT

The therapeutic electrical pulse delivery system may include a power source, a pulse generator, and a controller. The pulse generator may be operatively coupled to the power source. The pulse generator may include one or more capacitors. Each of the one or more capacitors may include a first electrode, a second electrode, and a dielectric disposed between the first electrode and the second electrode. The one or more capacitors may include an energy capacity at least 10 percent greater than a maximum energy of therapeutic electrical pulses delivered by the therapeutic electrical pulse delivery system. The controller may include one or more processors and may be operatively coupled to the power source or the pulse generator to charge the one or more capacitors and cause the pulse generator to deliver a therapeutic electrical pulse using the charged one or more capacitors.

This application claims the benefit of U.S. Provisional Patent Application Ser. No. 63/441,076, filed Jan. 25, 2023, the entire content of which is incorporated herein by reference.

### FIELD

The present disclosure relates to, among other things, therapeutic electrical pulse delivery systems and apparatus.

### TECHNICAL BACKGROUND

Therapeutic electrical pulse delivery systems and apparatus generally use capacitors to store energy and deliver a therapeutic pulse or shock to a patient. Capacitors may store energy in an electric field between two electrodes (e.g., a first electrode and a second electrode). Capacitors may discharge stored energy more rapidly than batteries or other power sources. Additionally, the energy stored by capacitors can have a higher voltage than energy stored and provided by typical batteries or other power sources while taking up less volume. Accordingly, capacitors may be used to provide high voltage pulses or shocks in therapeutic electrical pulse delivery systems and apparatus.

### BRIEF SUMMARY

As described herein, therapeutic electrical pulse delivery systems and apparatus with improved deformation factor, charge time, and longevity may be achieved using pulse generator capacitors with a greater energy capacity than required for therapeutic pulse delivery. A pulse generator of a therapeutic electrical pulse delivery system or apparatus may include one or more capacitors to store and deliver energy. The system or apparatus may be configured to deliver therapeutic electrical pulses at or below a maximum energy level using the pulse generator. The one or more capacitors may have an energy capacity at least 10 percent greater than the maximum energy level. Capacitors or capacitor banks that have higher energy capacities than required for delivery of therapeutic electrical pulses may have an improved deformation factor, charge time, and longevity than capacitors or capacitor banks that merely meet the energy capacity requirements for delivery of therapeutic electrical pulses. Accordingly, therapeutic electrical pulse delivery systems and apparatus described herein may have an improved deformation factor, decreased charge time, and increased longevity. Additionally, therapeutic electrical pulse delivery systems and apparatus described herein may have increased charging efficiency.

In one example, aspects of this disclosure relate to a therapeutic electrical pulse delivery system to deliver therapeutic electrical pulses. The therapeutic electrical pulse delivery system may include a power source, a pulse generator, and a controller. The pulse generator may be operatively coupled to the power source and may comprise one or more capacitors to store and deliver energy. The one or more capacitors may comprise an energy capacity at least 10 percent greater than a maximum energy of therapeutic electrical pulses delivered by the therapeutic electrical pulse delivery system. The controller may comprise one or more processors and may be operatively coupled to the power source or the pulse generator. The controller may be configured to charge the one or more capacitors of the pulse generator using the power source and cause the pulse generator to deliver a therapeutic electrical pulse using the charged one or more capacitors.

In another example, aspects of this disclosure relate to a therapeutic pulse generator to deliver therapeutic electrical pulses. The therapeutic pulse generator may include an input operatively couplable to a power source, one or more capacitors to store and deliver energy, and an output operatively coupled to the one or more capacitors to deliver a therapeutic electrical pulse using energy stored in the one or more capacitors. The one or more capacitors may comprise an energy capacity at least 10 percent greater than a maximum energy of therapeutic electrical pulses delivered by the therapeutic pulse generator.

In another example, aspects of this disclosure relate to an implantable medical device to deliver therapeutic electrical pulses. The implantable medical device may comprise a housing adapted to be implanted in a patient, a power source disposed in the housing, a pulse generator, and a controller. The pulse generator may be operatively coupled to the power source and may comprise one or more capacitors to store and deliver energy. The one or more capacitors may comprise an energy capacity at least 10 percent greater than a maximum energy of therapeutic electrical pulses delivered by the implantable medical device. The controller may comprise one or more processors and may be operatively coupled to the power source or the pulse generator. The controller configured to charge the one or more capacitors of the pulse generator using the power source and cause the pulse generator to deliver a therapeutic electrical pulse using the charged one or more capacitors.

Advantages and additional features of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### BRIEF DESCRIPTION OF THE DRAWINGS

The following detailed description of specific embodiments of the present disclosure can be best understood when read in conjunction with the following drawings, in which:

FIG. 1 is a conceptual drawing illustrating an embodiment of a therapeutic electrical pulse delivery system in conjunction with a patient;

FIG. 2 is a conceptual drawing illustrating another embodiment of a therapeutic electrical pulse delivery system in conjunction with the patient;

FIG. 3 is schematic block diagram of the electrical components of the therapeutic electrical pulse delivery systems of FIGS. 1 and 2;

FIG. 4 is schematic block diagram of a pulse generator of the therapeutic electrical pulse delivery systems of FIGS. 1-3.

The schematic drawing is not necessarily to scale.

### DETAILED DESCRIPTION

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

Generally, therapeutic electrical pulse delivery systems such as, for example, implantable cardioverter defibrillators take time to charge one or more capacitors of a pulse generator to ready therapy or therapeutic electrical pulse delivery. The time to charge may depend on factors such as efficiency of the capacitor and the deformed efficiency factor. The efficiency of a capacitor may be a ratio of delivered energy to input energy of the capacitor when the capacitor is new fresh capacitor. The deformed efficiency factor may be a ratio of input energy of a used capacitor to an input energy of a fresh capacitor. In general, as capacitors are used or otherwise age, the ratio of input energy of the used capacitor to the input energy of the capacitor when fresh increases. Accordingly, as capacitors are used or otherwise age, the deformation factor increases. In other words, older capacitors may require more energy to charge to the same level than new capacitors.

Typically, designs for therapeutic electrical pulse delivery systems seek to achieve smaller devices. In general, smaller capacitors have lower energy capacities than larger capacitors of the same type and design. Accordingly, capacitors chosen for therapeutic electrical pulse delivery systems and devices may have energy capacities that meet but do not greatly exceed operational requirements. For example, capacitors for therapeutic electrical pulse delivery may be chosen that have an energy capacity large enough to deliver therapeutic electrical pulses at a maximum energy that the device or system is configured to deliver without exceeding the energy requirements. A choice of capacitors that meet the operational energy requirements may allow for the use smaller capacitors.

However, it has been found that the use of capacitors with higher energy capacities for the same applications (e.g., the same therapeutic electrical pulse delivery parameters) may have a lower deformed efficiency factor than capacitors with lower energy capacities. Accordingly, the use of capacitors with higher energy capacity may result in less power needed to charge capacitors for therapeutic electrical pulse delivery over time. Furthermore, because less power is needed, the same power source may be able to charge the capacitors that have a higher energy capacity more quickly over time. Still further, because the deformation factor of the capacitors with higher energy capacities increases at a slower rate, the therapeutic electrical pulse delivery systems may last longer. In other words, therapeutic electrical pulse delivery systems and apparatus that include pulse generator capacitors with greater energy capacity than required for therapeutic pulse delivery as described herein, may allow for an improved deformation factor, charge time, longevity, and charging efficiency.

As used herein, the term “energy capacity” may refer to a nominal maximum energy that a device (e.g., a capacitor) may be charged to or otherwise store. For example, a 45-joule capacitor may have a nominal maximum energy capacity of 45 joules.

According to one aspect there is provided a therapeutic electrical pulse delivery system. The therapeutic electrical pulse delivery system may include a power source, a pulse generator, and a controller. The pulse generator may be operatively coupled to the power source. The pulse generator may include one or more capacitors. Each of the one or more capacitors may include a first electrode, a second electrode, and a dielectric disposed between the first electrode and the second electrode. The one or more capacitors may include an energy capacity at least 10 percent greater than a maximum energy of therapeutic electrical pulses delivered by the therapeutic electrical pulse delivery system. The controller may include one or more processors and may be operatively coupled to the power source or the pulse generator. The controller may be configured to charge the one or more capacitors of the pulse generator using the power source and cause the pulse generator to deliver a therapeutic electrical pulse using the charged one or more capacitors.

The pulse generator may include any suitable number of capacitors. In one or more embodiments, the one or more capacitors consists of a single capacitor. The use of capacitors with higher energy capacities may allow a single capacitor to have a maximum voltage and capacitance to deliver therapeutic electrical pulses at the desired voltage. The use of a single capacitor may allow the size and cost of pulse generators and therapeutic electrical pulse delivery systems to be reduced compared to apparatus and systems that use multiple electrolytic capacitors.

Although the pulse generator may consist of a single capacitor, arrangements that include multiple capacitors may allow for a variety of pulse generator arrangements and/or dynamic therapeutic electrical pulse delivery. The one or more capacitors may be arranged to be charged in parallel and to deliver the therapeutic electrical pulse in parallel. In other words, each of the one or more capacitors may be arranged such that a terminal of each capacitor is coupled to a first voltage node and the other terminal of each capacitor is coupled to a second voltage node. Capacitors arranged to deliver therapeutic electrical pulses in parallel may provide redundancy that allows the pulse generator to continue delivering therapeutic electrical pulses as long as one capacitor has not failed and can deliver the therapeutic electrical pulse.

In one or more embodiments, the one or more capacitors may be arranged to charge in parallel and to deliver the therapeutic electrical pulse in series. The pulse generator may include an input operatively coupled to the power source and configured to charge the one or more capacitors in parallel. The input may include one or more switches to allow power to be received from the power source to charge the one or more capacitors. The input may be controlled by the controller to charge the one or more capacitors. For example, the controller may be configured to open and close one or more switches of the input. The switches of the input may include, for example, one or more relays, transistors, digital switches, or other switch apparatus.

The pulse generator may further include an output operatively coupled to the one or more capacitors and configured to deliver the therapeutic electrical pulse from the one or more capacitors. The output may include one or more switches to allow therapeutic electrical pulses to be delivered using the one or more capacitors. The output may be controlled by the controller to deliver the therapeutic electrical pulses. For example, the controller may be configured to open and close one or more switches of the output. The switches of the output may include, for example, one or more relays, transistors, digital switches, or other switch apparatus.

In one or more embodiments, the output may be static such that the therapeutic electrical pulse is delivered from the capacitors in the same arrangement each time. In other words, the output may be configured to deliver the therapeutic electrical pulse from the capacitors in series, in parallel, or a in a combination of series and parallel. In one or more other embodiments, the output may be dynamic such that the therapeutic electrical pulse can be delivered from different combinations and arrangements of the one or more capacitors. In other words, the output may be configured to selectively deliver the therapeutic electrical pulse from the one or more capacitors in series, parallel, or in a combination of series and parallel.

For example, the output may include a plurality of switches that can be open or closed in various arrangements to deliver the therapeutic electrical pulse from the one or more capacitors. In one or more embodiments, a series of stepped-up therapeutic electrical pulses can be delivered by three capacitors. The series of stepped-up therapeutic electrical pulses may include a therapeutic electrical pulse delivered by the three capacitors in parallel. The series of stepped-up therapeutic electrical pulses may include another therapeutic electrical pulse delivered by two of the three capacitors in parallel with one another and the third of the three capacitors arranged in series with the two capacitors. The series of stepped-up therapeutic electrical pulses may include yet another therapeutic electrical pulse delivered by the three capacitors in series. Although three capacitors are used as an example, it will be appreciated that various arrangements of two capacitors or four or more capacitors can also be utilized.

The one or more capacitors may have any suitable maximum voltage to provide therapeutic electrical pulses. Each of the one or more capacitors may have an individual maximum voltage that can be the same or different from the individual maximum voltage of other capacitors of the one or more capacitors. At least one of the one or more capacitors may have a maximum voltage of, for example, at least 100 volts, at least 300 volts, at least 1000 volts, at least 2000 volts, or any voltage therebetween. In one or more embodiments, at least one capacitor of the one or more capacitors may have a maximum voltage of at least 1000 volts.

The one or more capacitors may have any suitable capacitance to provide therapeutic electrical pulses. In other words, the nominal capacitance of the one or more capacitors may depend on the parameters for the therapeutic electrical pulses to be delivered. Each of the one or more capacitors may have an individual capacitance that can be the same or different from the individual capacitance of other capacitors of the one or more capacitors. At least one of the one or more capacitors may have a capacitance of, for example, at least 40 Microfarads and no greater than 300 Farads or any capacitance therebetween. In one or more embodiments, the one or more capacitors may have a capacitance of at least 140 Microfarads and no greater than 160 Microfarads as seen from an output of the pulse generator. In other words, the total capacitance of the one or more capacitors as seen from the output of the pulse generator may be at least 140 Microfarads and no greater than 160 Microfarads.

The one or more capacitors may have any suitable energy capacity to store energy and deliver therapeutic electrical pulses. In other words, the nominal energy capacity of the one or more capacitors may depend on the parameters for the therapeutic electrical pulses to be delivered. Each of the one or more capacitors may have an individual energy capacity that can be the same or different from the individual energy capacity of other capacitors of the one or more capacitors. In general, the energy capacity of the one or more capacitors may be greater than a maximum energy of the therapeutic electrical pulses to be delivered. In other words, a total energy capacity of the one or more capacitors may exceed a maximum energy that the therapeutic electrical pulse delivery system or device is configured to deliver. The energy capacity of the one or more capacitors may be at least 10 percent greater, at least 15 percent greater, at least 20 percent greater, or at least 25 percent greater than the maximum energy of the therapeutic electrical pulses to be delivered.

The pulse generator may be configured to deliver the therapeutic electrical pulse with any suitable energy level or voltage. In general, the pulse generator may be configured to deliver the therapeutic electrical pulse with that an energy of at least 2 joules and no greater than 85 joules. For wearable devices, such as a vest, the pulse generator may be configured to deliver the therapeutic electrical pulse with that an energy up to 200 joules or up to 300 joules. The pulse generator may be configured to deliver the electrical pulse with a voltage of, for example, at least 100 volts, at least 300 volts, at least 1000 volts, at least 2000 volts, or any voltage therebetween. In one or more embodiments, the pulse generator may be configured to deliver the electrical pulse with a voltage of at least 1000 volts. The pulse generator may include a step-up converter to boost or increase the voltage received from the power source. The step-up converter may be configured to increase the voltage received from the power source to a therapeutic pulse voltage. An output voltage of the step-up converter may be adjustable based on a desired therapeutic pulse voltage. For example, the controller may be configured to adjust the output voltage of the step-up converter based on the desired therapeutic pulse voltage.

The therapeutic electrical pulse delivery system may include a housing. The pulse generator may be disposed in the housing. The housing may include a wearable device such as, a vest, a cuff, headwear, etc. In one or more embodiments, the housing may include a vest. The therapeutic electrical pulse delivery system may include any suitable medical device. In one or more embodiments, an implantable medical device includes the therapeutic electrical pulse delivery system.

According to one aspect there is provided a therapeutic pulse generator. The therapeutic pulse generator may include an input, one or more capacitors, and an output. The input may be operatively couplable to a power source. The one or more capacitors may be coupled to the input to receive and store energy provided by the power source. The one or more capacitors may include an energy capacity at least 10 percent greater than a maximum energy of therapeutic electrical pulses delivered by the therapeutic pulse generator. The output may be operatively coupled to the one or more capacitors to deliver a therapeutic electrical pulse using energy stored in the one or more capacitors.

The pulse generator may include any suitable number of capacitors. In one or more embodiments, the one or more capacitors consists of a single capacitor. The use of capacitors with higher energy capacities may allow a single capacitor to have a maximum voltage and capacitance to deliver therapeutic electrical pulses at the desired voltage. The use of a single capacitor may allow the size and cost of pulse generators to be reduced compared to apparatus and systems that use multiple electrolytic capacitors.

Although the pulse generator may consist of a single capacitor, arrangements that include multiple capacitors may allow for a variety of pulse generator arrangements and/or dynamic therapeutic electrical pulse delivery. The one or more capacitors may be arranged to be charged in parallel and to deliver the therapeutic electrical pulse in parallel. In other words, each of the one or more capacitors may be arranged such that a terminal of each capacitor is coupled to a first voltage node and the other terminal of each capacitor is coupled to a second voltage node. In one or more embodiments, the one or more capacitors may be arranged to charge in parallel and to deliver the therapeutic electrical pulse in series.

The input may be configured to charge the one or more capacitors in parallel. The input may include one or more switches to allow power to be received from the power source to charge the one or more capacitors. The input may be controllable to charge the one or more capacitors. For example, the one or more switches may be opened or closed by a controller to control charging of the one or more capacitors. The switches of the input may include, for example, one or more relays, transistors, digital switches, or other switch apparatus.

The output may be configured to deliver the therapeutic electrical pulse from the one or more capacitors. The output may include one or more switches to allow therapeutic electrical pulses to be delivered using the one or more capacitors. The output may be controllable by a controller to deliver the therapeutic electrical pulses. For example, one or more switches of the output may be opened or closed by a controller to control the delivery of the therapeutic electrical pulse. The switches of the output may include, for example, one or more relays, transistors, digital switches, or other switch apparatus.

In one or more embodiments, the output may be static such that the therapeutic electrical pulse is delivered from the capacitors in the same arrangement each time. In other words, the output may be configured to deliver the therapeutic electrical pulse from the capacitors in series, in parallel, or a in a combination of series and parallel. In one or more other embodiments, the output may be dynamic such that the therapeutic electrical pulse can be delivered from different combinations and arrangements of the one or more capacitors. In other words, the output may be configured to selectively deliver the therapeutic electrical pulse from the one or more capacitors in series, parallel, or in a combination of series and parallel.

For example, the output may include a plurality of switches that can be open or closed in various arrangements to deliver the therapeutic electrical pulse from the one or more capacitors. In one or more embodiments, a series of stepped-up therapeutic electrical pulses can be delivered by three capacitors. The series of stepped-up therapeutic electrical pulses may include a therapeutic electrical pulse delivered by the three capacitors in parallel. The series of stepped-up therapeutic electrical pulses may include another therapeutic electrical pulse delivered by two of the three capacitors in parallel with one another and the third of the three capacitors arranged in series with the two capacitors. The series of stepped-up therapeutic electrical pulses may include yet another therapeutic electrical pulse delivered by the three capacitors in series. Although three capacitors are used as an example, it will be appreciated that various arrangements of two capacitors or four or more capacitors can also be utilized.

The one or more capacitors may have any suitable maximum voltage to provide therapeutic electrical pulses. Each of the one or more capacitors may have an individual maximum voltage that can be the same or different from the individual maximum voltage of other capacitors of the one or more capacitors. At least one of the one or more capacitors may have a maximum voltage of, for example, at least 100 volts, at least 300 volts, at least 1000 volts, at least 2000 volts, or any voltage therebetween. In one or more embodiments, at least one capacitor of the one or more capacitors may have a maximum voltage of at least 1000 volts.

The one or more capacitors may have any suitable capacitance to provide therapeutic electrical pulses. In other words, the nominal capacitance of the one or more capacitors may depend on the parameters for the therapeutic electrical pulses to be delivered. Each of the one or more capacitors may have an individual capacitance that can be the same or different from the individual capacitance of other capacitors of the one or more capacitors. At least one of the one or more capacitors may have a capacitance of, for example, at least 40 Microfarads and no greater than 300 Farads or any capacitance therebetween. In one or more embodiments, the one or more capacitors may have a capacitance of at least 140 Microfarads and no greater than 160 Microfarads as seen from an output of the pulse generator. In other words, the total capacitance of the one or more capacitors as seen from the output of the pulse generator may be at least 140 Microfarads and no greater than 160 Microfarads.

The one or more capacitors may have any suitable energy capacity to store energy and deliver therapeutic electrical pulses. In other words, the nominal energy capacity of the one or more capacitors may depend on the parameters for the therapeutic electrical pulses to be delivered. Each of the one or more capacitors may have an individual energy capacity that can be the same or different from the individual energy capacity of other capacitors of the one or more capacitors. In general, the energy capacity of the one or more capacitors may be greater than a maximum energy of the therapeutic electrical pulses to be delivered. In other words, a total energy capacity of the one or more capacitors may exceed a maximum energy that the pulse generator is configured to deliver. The energy capacity of the one or more capacitors may be at least 10 percent greater, at least 15 percent greater, at least 20 percent greater, or at least 25 percent greater than the maximum energy of the therapeutic electrical pulses delivered by the pulse generator.

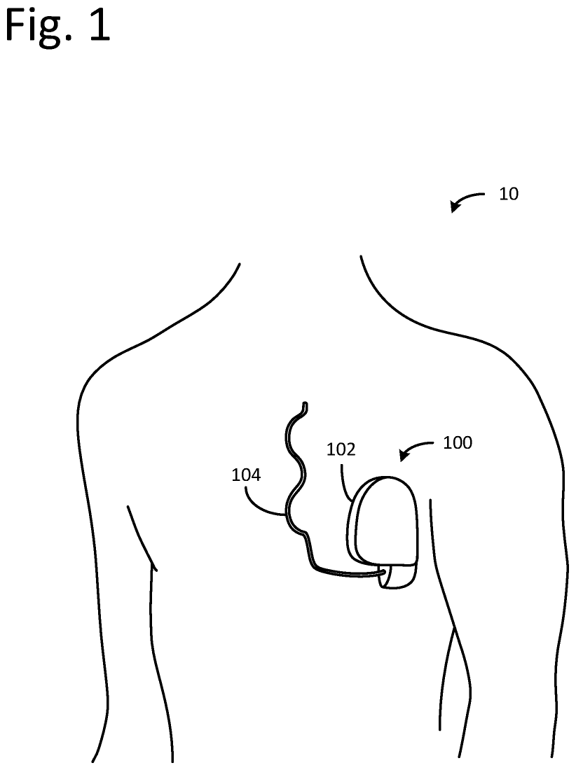

An embodiment of a therapeutic electrical pulse delivery system that includes capacitors with excess energy capacity as described herein, is depicted in FIG. 1. FIG. 1 shows a conceptual drawing illustrating the therapeutic electrical pulse delivery system 100 in conjunction with a patient 10. As depicted in FIG. 1, the therapeutic electrical pulse delivery system 100 includes a housing 102 that defines the exterior of an implantable medical device. The therapeutic electrical pulse delivery system 100 may be or may be included in, any suitable implantable medical device such as, for example, implantable pulse generators, implantable cardioverter defibrillators, implantable cardiac contractility modulators, implantable neurostimulators, implantable mechanical assist devices, etc.

The therapeutic electrical pulse delivery system 100 may also include one or more leads 104 to deliver therapeutic electrical pulses to desired treatment areas of the patient 10. The leads 104 may include one or more electrodes (not shown) to facilitate delivery of the therapeutic electrical pulses to the desired treatment areas. In one or more embodiments, the therapeutic electrical pulse delivery system 100 may include one or more electrodes without any leads. For example, when the therapeutic electrical pulse delivery system 100 can be implanted at the desired treatment area, leads may not be needed to deliver the therapeutic electrical pulses.

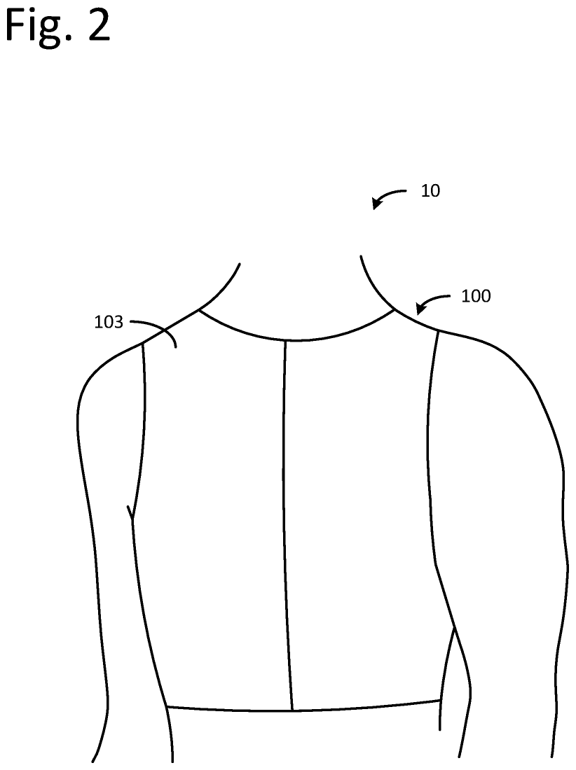

In addition to implantable medical devices, the therapeutic electrical pulse delivery system 100 may be, or may be housed in, wearable or other non-implantable devices. An example of the therapeutic electrical pulse delivery system 100 housed in a vest 103 is shown in FIG. 2. Other wearable devices may include cuffs, skin patches, etc. Wearable therapeutic electrical pulse delivery systems that use capacitors with excess energy capacity may have reduced charge times, increased charging efficiency, and greater longevity when compared to therapeutic electrical pulse delivery systems that use capacitors with little or no excess energy capacity.

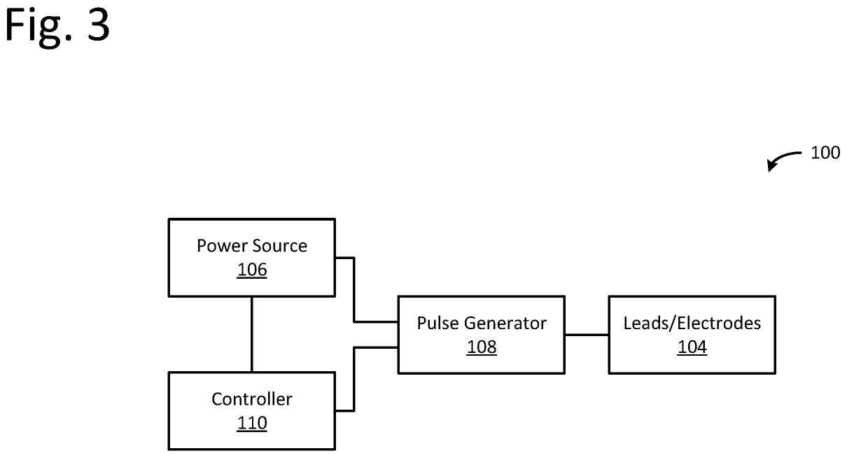

A schematic block diagram of the electronic components of the therapeutic electrical pulse delivery system 100 are depicted in FIG. 3. The electronic components of the therapeutic electrical pulse delivery system 100 can be disposed in the housing 102 to be implanted in the patient 10 as shown in FIG. 1 or disposed in a wearable device such as the vest 103 of FIG. 2. The therapeutic electrical pulse delivery system 100 may include a power source 106, a pulse generator 108, and a controller 110.

The power source 106 may be operatively coupled to the pulse generator 108 to provide energy to charge the pulse generator 108. In general, the power source 106 may be a voltage source. The power source 106 may be configured to provide energy at a voltage of at least 10 volts to no greater than 2000 volts. Accordingly, the current provided by the power source 106 to the pulse generator 108 may vary based on a state of charge of the pulse generator. The power source 106 may include any suitable energy storage and/or power delivery apparatus. The power source 106 may include one or more, for example, batteries, electrochemical cells, fuel cells, super capacitors, switches, controllers, battery management systems, or other energy storage and/or power delivery apparatus.

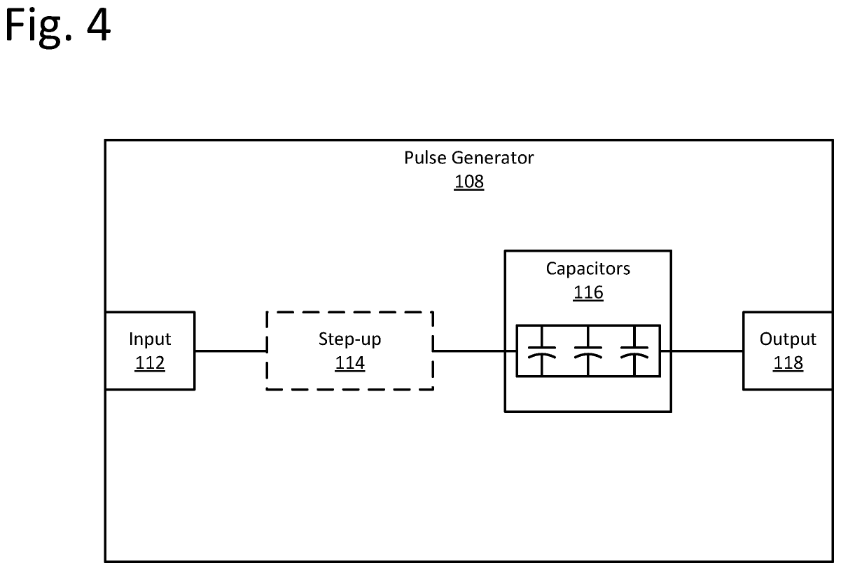

The pulse generator 108 may be operatively coupled to the power source 106. The pulse generator 108 may be configured to receive energy from the power source 106 within a nominal voltage range of the power source 106. A schematic block diagram of an embodiment of the pulse generator or therapeutic pulse generator 108 is depicted in FIG. 4. The pulse generator 108 may be operatively coupled to or operatively couplable to the power source 106 via an input 112. The input 112 may include a switch to allow control of the receipt of energy from the power source 106. Alternatively, the input 112 may be operatively coupled to an external switch. The external switch may be part of the pulse generator 108 generally or included in the power source 106. The switch, whether internal or external to the pulse generator, may include any suitable device or devices. The switch may include, for example, one or more transistors, electromechanical switches, toggles, etc.

The pulse generator 108 may include one or more capacitors 116. Each of the one or more capacitors 116 may include a first electrode, a second electrode, and a dielectric disposed between the first electrode and the second electrode. The one or more capacitors 116 may have an energy capacity greater than the maximum energy level of therapeutic electrical pulses delivered by the therapeutic electrical pulse delivery system 100. For example, the therapeutic electrical pulse delivery system 100 may be configured to supply 35 to 40 joules of energy per delivered therapeutic electrical pulse while the one or more capacitors 116 may have an energy capacity of at least 45 joules. In one or more embodiments, the one or more capacitors 116 may have an energy capacity at least 10 percent greater than a maximum energy of therapeutic electrical pulses delivered by the electrical pulse delivery system. In one or more embodiments, the one or more capacitors 116 may have an energy capacity at least 20 percent greater than a maximum energy of therapeutic electrical pulses delivered by the electrical pulse delivery system. In one or more embodiments, the one or more capacitors 116 may have an energy capacity at least 25 percent greater than a maximum energy of therapeutic electrical pulses delivered by the electrical pulse delivery system.

The one or more capacitors 116 may have any suitable energy capacity to provide a therapeutic electrical pulse at any suitable energy for a particular therapeutic electrical pulse delivery system. For example, some implantable therapeutic electrical pulse delivery systems may deliver therapeutic electrical up to 85 joules. Accordingly, the one or more capacitors 116 may have an energy capacity in a range of at least 93.5 joules up to 106.25 joules when the therapeutic electrical pulse delivery system 100 is or includes an implantable medical device. Further for example, some wearable or external therapeutic electrical pulse delivery systems may deliver therapeutic electrical pulses of up to 300 joules. Accordingly, the one or more capacitors 116 may have an energy capacity in a range of at least 330 joules up to 375 joules. In one embodiment, the one or more capacitors 116 may have an energy capacity of at least 40 joules to no greater than 50 joules.

The pulse generator 108 may include an output 118 operatively coupled to the one or more capacitors to deliver a therapeutic electrical pulse using energy stored in the one or more capacitors. The output 118 may include a switch to allow control delivery of the therapeutic electrical pulse from the one or more capacitors 116. The switch may include any suitable device or devices to control the delivery of the therapeutic electrical pulse. The switch may include, for example, one or more transistors, electromechanical switches, toggles, etc.

The pulse generator 108 may also include a step-up converter 114. The step-up converter 114 may be configured to increase the voltage of energy received from the power source 106. In one or more embodiments, the output voltage of the power source 106 may be lower than a desired voltage for delivery of a therapeutic electrical pulse. In such embodiments, the step-up converter 114 may boost the voltage provided by the power source 106. In one or more embodiments, the step-up converter 114 may be adjustable to allow the pulse generator 108 to deliver therapeutic electrical pulses at various voltages. For example, the step-up converter 114 may be configured to adjust an output voltage based on commands or signals received from the controller 110. The step-up converter 114 may include any suitable device or devices to boost or increase the voltage provided by the power source 106. The step-up converter 114 may include one or more of, for example, a direct current (DC)-DC converter, switches, transistors, transformers, inductors, etc.

Energy transfer from the power source 106 to the pulse generator 108 may be controlled by a controller 110. The controller 110 may be operatively coupled to the power source 106 and/or the pulse generator 108. The controller 110 may be configured to charge one or more capacitors 116 of the pulse generator 108 using the power source 106 and cause the pulse generator 108 to deliver a therapeutic electrical pulse using the charged one or more capacitors 116. To charge the one or more capacitors 116, the controller 110 may be configured to close a switch associated with the input 112 and/or the power source 106 to allow current to flow from the power source 106 to the pulse generator 108. To deliver the therapeutic electrical pulse, the controller 110 may be configured to close a switch associated with the output 118 of the pulse generator 108. Additionally, the controller 110 may be configured to open the switch of the input 112 or the power source 106 before closing the switch of the output 118. When the switch of the output 118 is closed, energy stored in the capacitors 116 may flow through the output to one or more leads/electrodes 104 and ultimately to therapy delivery sites of the patient 10.

The controller 110 may include any suitable analogue or digital circuitry to charge the one or more capacitors 116 and deliver therapeutic electrical pulses. The controller may include, for example, one or more processors, logic gates, operational amplifiers, transistors, analogue-to-digital converters, sensors, or other circuitry or devices to control the power source 106 and/or the pulse generator 108. The controller 110 may include data storage for data storage and access to processing programs or routines that may be employed to carry out the techniques, processes, and algorithms for charging the one or more capacitors 116 and delivering a therapeutic electrical pulse. For example, processing programs or routines may include programs or routines for pulse delivery timing, pulse delivery triggers, opening and closing switches, determining an output voltage, adjusting an output voltage, filtering background noise, computational mathematics, matrix mathematics, Fourier transforms, compression algorithms, calibration algorithms, inversion algorithms, signal processing algorithms, normalizing algorithms, deconvolution algorithms, averaging algorithms, standardization algorithms, comparison algorithms, vector mathematics, or any other processing required to implement one or more embodiments as described herein.

The controller 110 may also include a communication interface to communicate with one or more external devices. The communication interface may include any suitable hardware or devices to provide wired or wireless communication with the one or more external devices. For example, the communication interface may include one or more of a receiver, transmitter, transceiver, ethernet port, Universal Serial Bus (USB) port, cables, controller, or other device to facilitate wired or wireless communication. The communication interface may facilitate communication using any suitable protocol or protocols. For example, the communication interface may utilize Ethernet, Recommended Standard 232, Universal Asynchronous Receiver Transmitter or Universal Synchronous Asynchronous Receiver Transmitter (UART/USART), USB, BLUETOOTH, Wi-Fi, Near Field Communication (NCF), etc. The communication interface may allow communication between the therapeutic electrical pulse delivery system 100 and a computing apparatus.

The invention is defined in the claims. However, below there is provided a non-exhaustive list of non-limiting examples. Any one or more of the features of these examples may be combined with any one or more features of another example, embodiment, or aspect described herein.

Example Ex1: A therapeutic electrical pulse delivery system to deliver therapeutic electrical pulses, the therapeutic electrical pulse delivery system comprising: a power source; a pulse generator operatively coupled to the power source, the pulse generator comprising one or more capacitors to store and deliver energy, the one or more capacitors comprising an energy capacity at least 10 percent greater than a maximum energy of therapeutic electrical pulses delivered by the therapeutic electrical pulse delivery system; and a controller comprising one or more processors and operatively coupled to the power source or the pulse generator, the controller configured to: charge the one or more capacitors of the pulse generator using the power source; and cause the pulse generator to deliver a therapeutic electrical pulse using the charged one or more capacitors.

Example Ex2: The therapeutic electrical pulse delivery system as in example Ex1, wherein the pulse generator further comprises: an input operatively coupled to the power source and configured to charge the one or more capacitors in parallel; and an output operatively coupled to the one or more capacitors and configured to selectively deliver the therapeutic electrical pulse from the one or more capacitors in series, parallel, or in a combination of series and parallel.

Example Ex3: The therapeutic electrical pulse delivery system as in any one of the previous examples, wherein the one or more capacitors consists of a single capacitor.

Example Ex4: The therapeutic electrical pulse delivery system as in any one of the previous examples, wherein the one or more capacitors has a maximum energy capacity of at least 40 joules and no greater than 50 joules.

Example Ex5: The therapeutic electrical pulse delivery system as in any one of the previous examples, wherein the pulse generator is configured to deliver the therapeutic electrical pulse comprising at least 30 joules and no greater than 40 joules.

Example Ex6: The therapeutic electrical pulse delivery system as in any one of the previous examples, wherein the one or more capacitors comprises an energy capacity at least 20 percent greater than a maximum energy of the therapeutic electrical pulses delivered by the therapeutic electrical pulse delivery system.

Example Ex7: The therapeutic electrical pulse delivery system as in any one of the previous examples, further comprising a housing, wherein the pulse generator is disposed in the housing.

Example Ex8: The therapeutic electrical pulse delivery system as in example Ex7, wherein the housing comprises a vest.

Example Ex9: A therapeutic pulse generator to deliver therapeutic electrical pulses, the therapeutic pulse generator comprising: an input operatively couplable to a power source; one or more capacitors to store and deliver energy, the one or more capacitors comprising an energy capacity at least 10 percent greater than a maximum energy of therapeutic electrical pulses delivered by the therapeutic pulse generator; and an output operatively coupled to the one or more capacitors to deliver a therapeutic electrical pulse using energy stored in the one or more capacitors.

Example Ex10: The therapeutic pulse generator as in example Ex9, wherein the input is configured to charge the one or more capacitors in parallel and the output is configured to selectively deliver the therapeutic electrical pulse from the one or more capacitors in series, parallel, or in a combination of series and parallel.

Example Ex11: The therapeutic pulse generator as in any one of examples Ex9 or Ex10, wherein the one or more capacitors consists of a single capacitor.

Example Ex12: The therapeutic pulse generator as in any one of examples Ex9 to Ex11, wherein the one or more capacitors comprises a plurality of capacitors in a stacked arrangement.

Example Ex13: The therapeutic pulse generator as in any one of examples Ex9 to Ex12, wherein the one or more capacitors has a maximum energy capacity of 40 joules and no greater than 50 joules.

Example Ex14: The therapeutic pulse generator as in any one of examples Ex9 to Ex13, wherein the pulse generator is configured to deliver the therapeutic electrical pulse comprising at least 30 joules and no greater than 40 joules.

Example Ex15: The therapeutic pulse generator as in any one of examples Ex9 to Ex15, wherein the one or more capacitors comprises an energy capacity at least 20 percent greater than a maximum energy of the therapeutic electrical pulses delivered by the therapeutic pulse generator.

Example Ex16: The therapeutic pulse generator as in any one of examples Ex9 to Ex15, further comprising a step-up converter operatively coupled to the input and the one or more capacitors to step-up a voltage received from the power source.

Example Ex17: An implantable medical device to deliver therapeutic electrical pulses, the implantable medical device comprising: a housing adapted to be implanted in a patient; a power source disposed in the housing; a pulse generator operatively coupled to the power source, the pulse generator comprising one or more capacitors to store and deliver energy, the one or more capacitors comprising an energy capacity at least 10 percent greater than a maximum energy of therapeutic electrical pulses delivered by the implantable medical device; and a controller comprising one or more processors and operatively coupled to the power source or the pulse generator, the controller configured to: charge the one or more capacitors of the pulse generator using the power source; and cause the pulse generator to deliver a therapeutic electrical pulse using the charged one or more capacitors.

Example Ex18: The implantable medical device as in example Ex17, wherein the pulse generator further comprises: an input operatively coupled to the power source and configured to charge the one or more capacitors in parallel; and an output operatively coupled to the one or more capacitors and configured to selectively deliver the therapeutic electrical pulse from the one or more capacitors in series, parallel, or in a combination of series and parallel.

Example Ex19: The implantable medical device as in any one of examples Ex17 or Ex18, wherein the one or more capacitors consists of a single capacitor.

Example Ex20: The implantable medical device as in any one of examples Ex17 to Ex19, wherein the one or more capacitors has a maximum energy capacity of at least 40 joules and no greater than 50 joules.

Example Ex21: The implantable medical device as in any one of examples Ex17 or Ex20, wherein the pulse generator is configured to deliver the therapeutic electrical pulse comprising at least 30 joules and no greater than 40 joules.

Example Ex22: The implantable medical device as in any one of examples Ex17 or Ex21, wherein the one or more capacitors comprises an energy capacity at least 20 percent greater than a maximum energy of the therapeutic electrical pulses delivered by the implantable medical device.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all of the listed elements or a combination of any two or more of the listed elements.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

1. A therapeutic electrical pulse delivery system to deliver therapeutic electrical pulses, the therapeutic electrical pulse delivery system comprising:
a power source;
a pulse generator operatively coupled to the power source, the pulse generator comprising one or more capacitors to store and deliver energy, the one or more capacitors comprising an energy capacity at least 10 percent greater than a maximum energy of therapeutic electrical pulses delivered by the therapeutic electrical pulse delivery system; and
a controller comprising one or more processors and operatively coupled to the power source or the pulse generator, the controller configured to:
charge the one or more capacitors of the pulse generator using the power source; and
cause the pulse generator to deliver a therapeutic electrical pulse using the charged one or more capacitors.

2. The therapeutic electrical pulse delivery system as in claim 1, wherein the pulse generator further comprises:
an input operatively coupled to the power source and configured to charge the one or more capacitors in parallel; and
an output operatively coupled to the one or more capacitors and configured to selectively deliver the therapeutic electrical pulse from the one or more capacitors in series, parallel, or in a combination of series and parallel.

3. The therapeutic electrical pulse delivery system as in claim 1, wherein the one or more capacitors consists of a single capacitor.

4. The therapeutic electrical pulse delivery system as in claim 1, wherein the one or more capacitors comprises an energy capacity at least 20 percent greater than a maximum energy of the therapeutic electrical pulses delivered by the therapeutic electrical pulse delivery system.

5. The therapeutic electrical pulse delivery system as in claim 1, further comprising a housing, wherein the pulse generator is disposed in the housing.

6. A therapeutic pulse generator to deliver therapeutic electrical pulses, the therapeutic pulse generator comprising:
an input operatively couplable to a power source;
one or more capacitors to store and deliver energy, the one or more capacitors comprising an energy capacity at least 10 percent greater than a maximum energy of therapeutic electrical pulses delivered by the therapeutic pulse generator; and
an output operatively coupled to the one or more capacitors to deliver a therapeutic electrical pulse using energy stored in the one or more capacitors.

7. The therapeutic pulse generator as in claim 6, wherein the input is configured to charge the one or more capacitors in parallel and the output is configured to selectively deliver the therapeutic electrical pulse from the one or more capacitors in series, parallel, or in a combination of series and parallel.

8. The therapeutic pulse generator as in claim 6, wherein the one or more capacitors consists of a single capacitor.

9. The therapeutic pulse generator as in claim 6, wherein the one or more capacitors comprises a plurality of capacitors in a stacked arrangement.

10. The therapeutic pulse generator as in claim 6, further comprising a step-up converter operatively coupled to the input and the one or more capacitors to step-up a voltage received from the power source.

11. An implantable medical device to deliver therapeutic electrical pulses, the implantable medical device comprising:
a housing adapted to be implanted in a patient;
a power source disposed in the housing;
a pulse generator operatively coupled to the power source, the pulse generator comprising one or more capacitors to store and deliver energy, the one or more capacitors comprising an energy capacity at least 10 percent greater than a maximum energy of therapeutic electrical pulses delivered by the implantable medical device; and
a controller comprising one or more processors and operatively coupled to the power source or the pulse generator, the controller configured to:
charge the one or more capacitors of the pulse generator using the power source; and
cause the pulse generator to deliver a therapeutic electrical pulse using the charged one or more capacitors.

12. The implantable medical device as in claim 11, wherein the pulse generator further comprises:
an input operatively coupled to the power source and configured to charge the one or more capacitors in parallel; and
an output operatively coupled to the one or more capacitors and configured to selectively deliver the therapeutic electrical pulse from the one or more capacitors in series, parallel, or in a combination of series and parallel.

13. The implantable medical device as in claim 11, wherein the one or more capacitors consists of a single capacitor.

14. The implantable medical device as in claim 11, wherein the one or more capacitors comprises an energy capacity at least 20 percent greater than a maximum energy of the therapeutic electrical pulses delivered by the implantable medical device.

15. The implantable medical device of claim 12, further comprising a step-up converter operatively coupled to the input and the one or more capacitors to step-up a voltage received from the power source.

16. The implantable medical device as in claim 11, wherein the one or more capacitors has a maximum energy capacity of at least 40 joules and no greater than 50 joules.

17. The implantable medical device as in claim 11, wherein the pulse generator is configured to deliver the therapeutic electrical pulse comprising at least 30 joules and no greater than 40 joules.

18. The implantable medical device of claim 11, wherein the one or more capacitors comprises a plurality of capacitors in a stacked arrangement.
